(19) United States
(12) Patent Application Publication
(10) Pub. No.: US 2023/0288420 A1
(43) Pub. Date: Sep. 14, 2023

(54) VERTICAL FLOW IMMUNOCHROMATOGRAPHIC ASSAY DEVICE AND METHOD OF USING THE SAME

(71) Applicant: Zeus Diagnostics LLC, Severna Park, MD (US)

(72) Inventor: Raouf A. Guirguis, Severna Park, MD (US)

(21) Appl. No.: 18/120,656

(22) Filed: Mar. 13, 2023

Related U.S. Application Data
(60) Provisional application No. 63/319,449, filed on Mar. 14, 2022.

(51) Int. Cl. G01N 33/569; G01N 33/543; G01N 1/38; A61B 10/00

(52) U.S. Cl. CPC G01N 33/56983; A61B 10/0051; G01N 1/38; G01N 33/54391; A61B 2010/0003; G01N 2001/383; G01N 2469/10

(57) ABSTRACT

A method and kit for detecting SARS-CoV-2 virus antigen present in an oropharyngeal lavage includes swishing and gargling a mouth rinse to distribute the same around the inside of the oral cavity and throughout an oropharyngeal space of the user. An absorbent end of a test stick is inserted into the oral cavity to collect liquid contents therefrom, the liquid contents including the mouth rinse and particles retrieved therefrom. The absorbent stick is removed and positioned vertically in a support stand while results are determined. Alternatively, the liquid contents are deposited from the oral cavity into a collection device, and the absorbent end of the test stick is inserted into the collection device and positioned vertically such that the test stick is retained in the collection device with the absorbent end of the test stick facing downwardly. An indicator on the test stick displays whether SARS-CoV-2 virus antigen is detected.

### CROSS REFERENCE TO RELATED APPLICATIONS

This application claims the benefit of U.S. Provisional Application No. 63/319,449, filed Mar. 14, 2022, which is incorporated by reference in its entirety.

### FIELD

The disclosure relates to methods and devices for rapidly detecting one or more analytes in a biological sample. More particularly, a rapid in-vitro immunochromatographic assay for the rapid qualitative detection of a virus, such as SARS-CoV-2 virus, antigen present in oropharyngeal lavage.

### BACKGROUND

SARS-CoV-2 is single-stranded RNA virus with envelope, the virion is approximately 50-200 nanometers in diameter. It has four structural proteins, known as the spike (S), envelope (E), membrane (M), and nucleocapsid (N) proteins; the N protein holds the RNA genome, and the S, E, and M proteins together create the viral envelope. The incubation period for COVID-19 typically ranges from 2 to 14 days. Those infected with the virus may be asymptomatic or develop common respiratory symptoms, including fever, cough and fatigue (other symptoms may include muscle pain, diarrhea, sore throat, loss of taste and smell, and abdominal pain). Severe patients may progress to acute respiratory distress syndrome (ARDS), septic shock, diffuse alveolar damage (DAD) and even death. Though the conventional nasopharyngeal test has provided a very useful tool for the detection of SARS-CoV-2 infection, the stringent sample collection requirement has limited its usage to professional or trained person.

Meanwhile, based on the study observation, oral mucosa and saliva glands are also reservoirs of SARS-CoV-2 during infection.

As can be seen, there is a need for a rapid in-vitro immunochromatographic assay for the qualitative detection of SARS-CoV-2 virus antigen present in human oropharyngeal lavage. The device and method embodied in the present invention makes the virus detection easier to perform and more accessible to non-trained person. The present invention, colloquially known as OroPharyngeal Lavage™ (OPL™) COVID-19 Antigen Test″, provides a quick and easy to use test to help in the diagnosis of SARS-CoV-2 infection to humans from oropharyngeal cavity samples.

The test is intended for laboratory, point of care, and home use as an aid in the rapid diagnosis of SARS-CoV-2 virus infections. According to the CDC’s guidance, COVID-19 antigen test has the best sensitivity from day 1 to day 5 after the onset of symptoms. Antigen levels in specimens collected beyond 5-7 days of the onset of symptoms may drop below the limit of detection of the test.

Conventional SARS-CoV-2 tests rely on swabbing the nasal cavity of an individual. The use of nasal swabs induces irritation and sneezing. This reaction from testing is problematic as it may spread SARS-CoV-2 during conventional testing procedures. Therefore, a need exists for reducing the spread of SARS-CoV-2 in testing procedures.

SARS-CoV-2 and at least one of its variants tends to reside in the oral cavity of an individual. Particularly, at least one of the variants resides more in the oral cavity than in the nasal cavity. There is a need for a testing method that quickly detects the presence of SARS-CoV-2 virus infections using the oral cavity in addition to or instead of the nasal cavity.

Therefore, a need exists for incorporating a test that can quickly detect the presence of SARS-CoV-2 virus infections in an individual. A need also exists for incorporating a test that is minimally invasive.

### SUMMARY

According to an embodiment, a method of detecting SARS-CoV-2 virus antigen present in an oropharyngeal lavage includes abstaining from placing liquid or food in an oral cavity of a user before collecting a sample; pouring a non-alcoholic mouth rinse into the oral cavity, and swishing and gargling the non-alcoholic mouth rinse for a first period of time to distribute the non-alcoholic mouth rinse through the inside of the oral cavity and throughout an oropharyngeal space of the user; inserting an absorbent end of a test stick into the oral cavity for a second period of time to collect liquid contents from the oral cavity, the liquid contents including the non-alcoholic mouth rinse and particles retrieved from the non-alcoholic mouth rinse; removing the absorbent end of the test stick from the oral cavity and covering the absorbent end with a cap; positioning the test stick in a Flip-Flop vertical position for a third period of time wherein the absorbent end of the test stick is facing upwardly; and displaying an indicator on the test stick that confirms whether SARS-CoV-2 virus antigen is detected in the oropharyngeal lavage.

According to another embodiment, a method of detecting SARS-CoV-2 virus antigen present in an oropharyngeal lavage includes abstaining from placing liquid or food in an oral cavity of a user before collecting a sample; pouring a non-alcoholic mouth rinse into the oral cavity, and swishing and gargling the non-alcoholic mouth rinse for a first period of time such that the non-alcoholic mouth rinse moves throughout both the oral cavity and an oropharyngeal space of the user; depositing a volume of liquid contents from the oral cavity, wherein the volume of liquid contents include the non-alcoholic mouth rinse and particles retrieved from the non-alcoholic mouth rinse, into a collection device; inserting an absorbent end of a test stick into the collection device for a second period and positioning the test stick in a vertical position such that the test stick is retained in the collection device for a third period of time wherein the absorbent end of the test stick is facing downwardly; and displaying an indicator on the test stick that confirms whether SARS-CoV-2 virus antigen is detected in the oropharyngeal lavage.

According to a further embodiment, a kit for detecting SARS-CoV-2 virus antigen present in an oropharyngeal lavage includes a test stick having a first end and a second end; a non-alcoholic mouth rinse; an absorbent tab disposed at the first end and configured to be inserted into an oral cavity of a user and configured to absorb at least the non-alcoholic mouth rinse and contents of the oral cavity of the user; a removable cap configured to cover said absorbent tab and configured to keep the absorbent tab sterile and dry until said removable cap is removed; a cartridge connected to said absorbent tab and configured to detect the SARS-CoV-2 virus antigen after the non-alcoholic mouth rinse is combined with contents from the oral cavity of the user and absorbed by the absorbent tab; and a stand having an opening configured to receive the second end of the test stick after the combined non-alcoholic mouth rinse and contents from the oral cavity of the user have been discharged onto the absorbent tab.

According to another embodiment, a kit for detecting SARS-CoV-2 virus antigen present in oropharyngeal lavage, includes a test stick having a first end and a second end wherein the first end is configured to be covered by a removable cap to preserve the sterility of the first end until the removable cap is removed from the first end; a non-alcoholic mouth rinse; an absorbent tab disposed at the first end and attached to a cartridge disposed within the test stick, said cartridge configured to detect the SARS-CoV-2 virus antigen after a volume including at least the non-alcoholic mouth rinse combined with contents from the oral cavity of the user; and a collection device having an opening configured to receive at least the volume of the non-alcoholic mouth rinse and contents from the oral cavity of the user, and configured to receive the absorbent tab such that the absorbent tab is submerged in the volume and retained in the collection device when the first end of the test stick is inserted into the collection device.

In all of the embodiments, a non-alcoholic mouth rinse may be used to detect and disinfect one or more analytes in the oropharyngeal lavage of a user, such as SARS-CoV-2 virus. The mouth rinse is able to break down the virus in the mucus of the user. The mouth rinse helps to produce virus particles by solubilizing the virus and opening the virus because the mouth rinse breaks down the virus such that the particles are able to be collected in a sample for testing.

The disclosure provides a method and device for rapidly detecting one or more analytes in the oropharyngeal lavage of a user, such as SARS-CoV-2 virus, Herpes simplex virus, Epstein barr virus, Cytomegalovirus, Moraxella catarrhalis, Norocardia sp, Streptococcus mutans, Streptococcus oralis, Eikenella sp. Neisseria sp, and Streptococcus salivarius. Advantageously, the disclosure provides a method and device for rapidly testing and detecting. In one embodiment, illustrated in FIGS. 1-5, test results may be obtained in as few as five minutes due to the inverting of the test stick after the absorbent end is exposed to the contents of the user’s mouth. In another embodiment, illustrated in FIGS. 6-9, test results may be obtained in as few as ten minutes when the absorbent end of the test stick is immersed in the contents of the user’s mouth and retained in a collection device.

Moreover, in addition to detecting the presence of one of the above, the method and device also provides a way to test whether a person is infected or not, whether a person is immunized or not, and whether a person is immuzined and infected.

The disclosure further provides a method and device that rapidly tests a fluid sample for the presence or absence of at least one analyte, such as those listed in the foregoing, and secures a separate fluid sample for later confirmation, and provides positive identification of an individual associated with the sample.

These and other features of the methods and kits of the subject disclosure will become more readily apparent to those skilled in the art from the following detailed description of the preferred embodiments taken in conjunction with the drawings.

### BRIEF DESCRIPTION OF DRAWINGS

The following descriptions should not be considered limiting in any way. With reference to the accompanying drawings, like elements are numbered alike:

FIG. 1 depicts a flowchart of a general method according to an exemplary embodiment;

FIG. 2 depicts a kit according to an exemplary embodiment;

FIG. 2a is a pamphlet of the kit of FIG. 2;

FIG. 3 depicts another view of the kit of FIG. 2 according to an exemplary embodiment;

FIG. 4 depicts an absorbent end of the test stick of FIGS. 2 and 3 with the cover removed;

FIG. 5 depicts the test stick of FIGS. 2-4 disposed vertically for testing;

FIG. 6 depicts a flowchart of a general method according to an exemplary embodiment;

FIG. 7 depicts a kit in accordance with an exemplary embodiment;

FIG. 8 depicts the kit of FIGS. 6-7 with the collection device removed from the protective covering shown in FIG. 7;

FIG. 9 depicts the collection device of FIGS. 6-8 with the user spitting into the collection device to be used for testing; and

FIG. 10 depicts a kit in accordance with an exemplary embodiment.

### DETAILED DESCRIPTION

A detailed description of one or more embodiments of the disclosed method and device is presented herein by way of exemplification and not limitation with reference to the Figures.

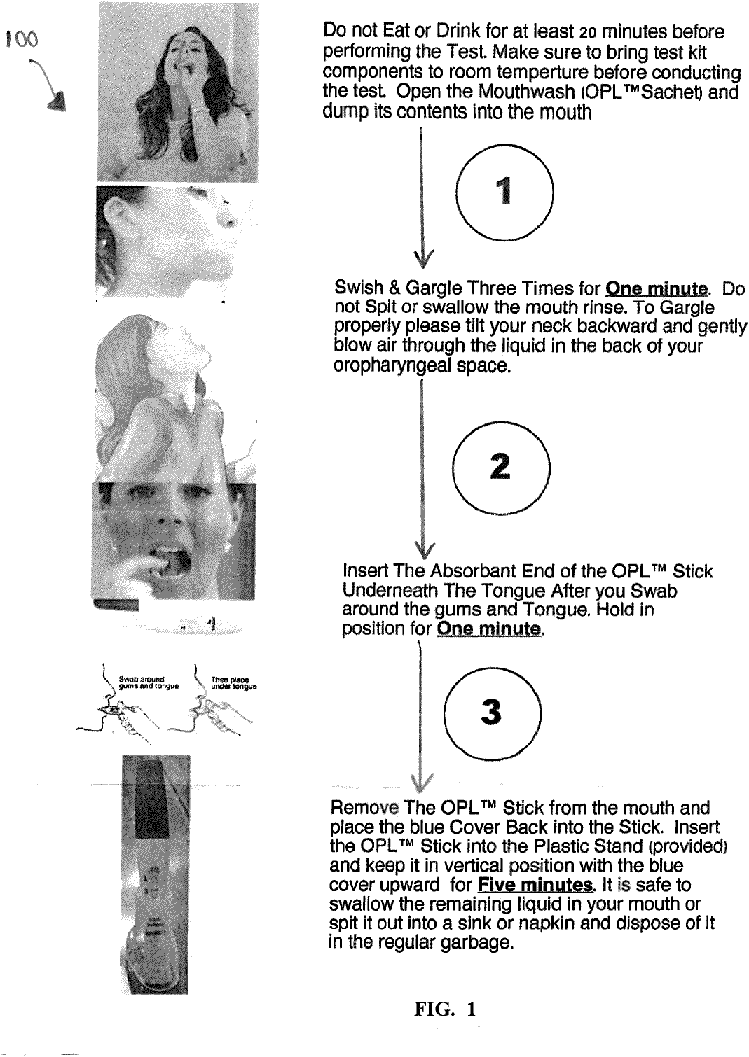

The present disclosure relates to a method of detecting SARS-CoV-2 virus antigen present in an oropharyngeal lavage. As seen in FIG. 1, the method 100 includes first abstaining from all liquids and foods for at least twenty minutes before performing the test. No food or liquid should be placed in an oral cavity of a user for a period of time before collecting a sample. The test kit components 150 should be brought to room temperature before conducting the test. After that pretest preparation time period, the user should pour a non-alcoholic mouth rinse into their mouth/oral cavity and move the mouth rinse around the oral cavity by swishing and gargling for a first period of time, for example, one minute, in order to thoroughly distribute the mouth rinse around the inside of the oral cavity and throughout an oropharyngeal space of the user. The mouth rinse should not be spit out of the user’s mouth or swallowed, but gargled when the user tilts back and gently blows air through the liquid in the back of the oropharyngeal space.

The mouth rinse that is used for testing may be, for example, OroPharyngeal Lavage™ (OPL™) . The OPL™ liquid in addition to being used to break up and gather virus containing particles from a user’s mouth is able to disinfect the sample. Test results have shown that 99.99% of virus samples have been disinfected. Advantageously, this reduces the spread of the SARS-CoV-2 infection between individuals during testing procedures.

In at least one SARS-CoV-2 variant (Omicron variant), it is more effective to test oral cavity fluids and collect saliva swabs for detection of the virus rather than nasal samples. Oral cavity fluids antigen test is a more effective method of testing for the presence of at least SARS-CoV-2 and SARS-CoV-2 variants than testing using nasal swabs. An Oral cavity fluids testing method is more sensitive and effective than nasal sampling. Particularly, with testing for the presence of SARS-CoV-2 variant Omicron, for example, saliva and oral washing using the OPL mouth rinse, for example, is effective and beneficial because the SARS-CoV-2 variant Omicron has been shown to prefer to reside in the oral cavity and colonize the oral cavity. Thus, it would be advantageous to use the OPL mouth rinse to collect samples from the oral cavity. Furthermore, it would be beneficial to have a testing method such as that disclosed herein because of the ability of the OPL mouth rinse to also disinfect the oral cavity and therefore reduce the amount of particles containing SARS-CoV-2 variant Omicron, thus, aiding to prevent the spread of SARS-CoV-2 variant Omicron.

Moreover, the OPL mouth rinse, for example, is used to break up the contents of the user’s mouth and interact with saliva in order to activate contents throughout the inside of the mouth, including the inside of the cheeks, the tongue and back of the throat, and to absorb the mouth rinse. The mouth rinse may be any non-alcoholic mouth rinse used to break up the contents of a user’s mouth and essentially aid in breaking up the bacterial components of a user’s mouth in order to detect the presence of various markers, e.g., SARS-CoV-2 virus, Herpes simplex virus, Epstein barr virus, Cytomegalovirus, Moraxella catarrhalis, Norocardia sp, Streptococcus mutans, Streptococcus oralis, Eikenella sp. Neisseria sp, and Streptococcus salivarius.

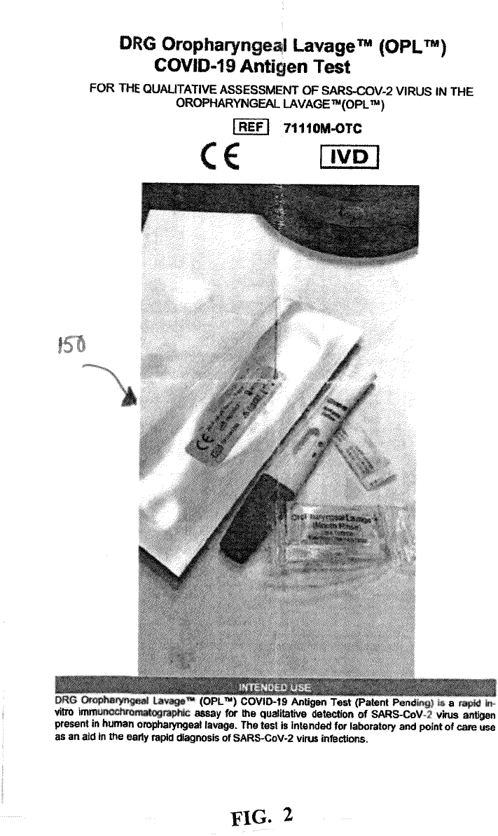

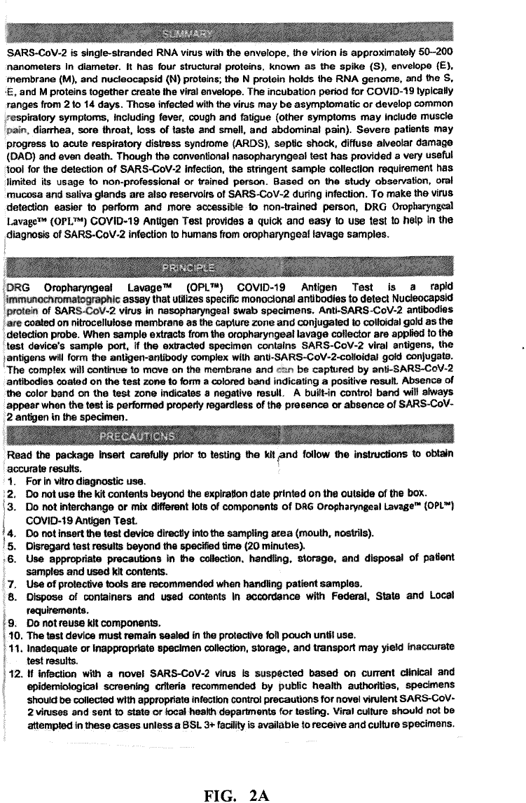

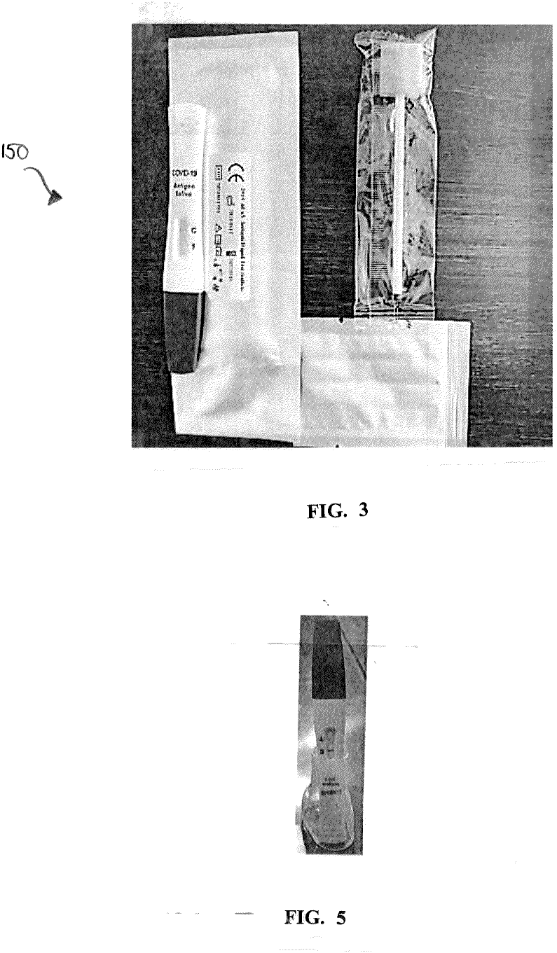

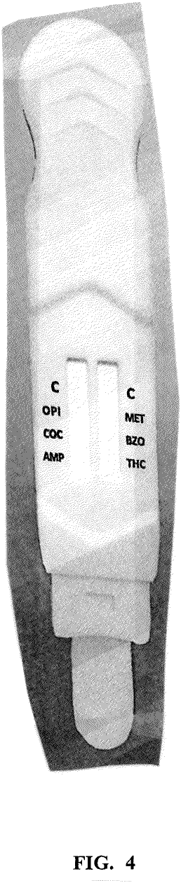

Next, a test stick shown in FIGS. 2-4 having first and second ends is used to collect contents from the user’s mouth. Specifically, an absorbent end disposed at the first end of the test stick is inserted into the user’s mouth, underneath the tongue, and help in position for a third time period, such as one minute. The test stick, as seen in FIG. 4, has an absorbent tab or surface configured for collecting at least a liquid is disposed at the first end and is configured to be inserted into an oral cavity of the user and configured to absorb at least the mouth rinse and contents of the oral cavity of the user. The user holds the test stick in place for at least one minute without biting, chewing, or moving the absorbent end. The absorbent end soaks the liquid contents from the user’s mouth that also include the particles extracted from the oropharyngeal space with the mouth rinse.

After the period of time for absorbing the contents from the user’s mouth on the absorbent end, the test stick is then removed from the oral cavity and the end is covered with a protective cap, as seen in FIG. 5. The user may safely swallow or spit out the remaining contents in their mouth that were not absorbed by the test stick. The protective cap is used to keep the absorbent end of the test stick sterile for insertion into the user’s mouth. Next, the test stick is positioned vertically for a fourth period of time, such as five minutes and retained in a vertical position. The stick may be placed into a plastic stand that is sized to retain the test stick in a vertical position, such as that illustrated in FIG. 5.

As seen in FIG. 5, the test stick is positioned such that the absorbent end is facing upwardly. The stick is positioned for a fourth period of time in order to register the results of the test. Once the test results are completed, an indicator reflecting the results of what is being tested for (e.g, SARS-CoV-2 presence, SARS-CoV-2 immunization), the results are displayed on the test stick. In the example illustrated in FIG. 5, the test stick confirms whether SARS-CoV-2 virus antigen is detected in the oropharyngeal lavage. The results are able to be displayed with a single marker such as a line adjacent an indicator on the test stick indicating whether the test results are positive or negative for the antigen being tested.

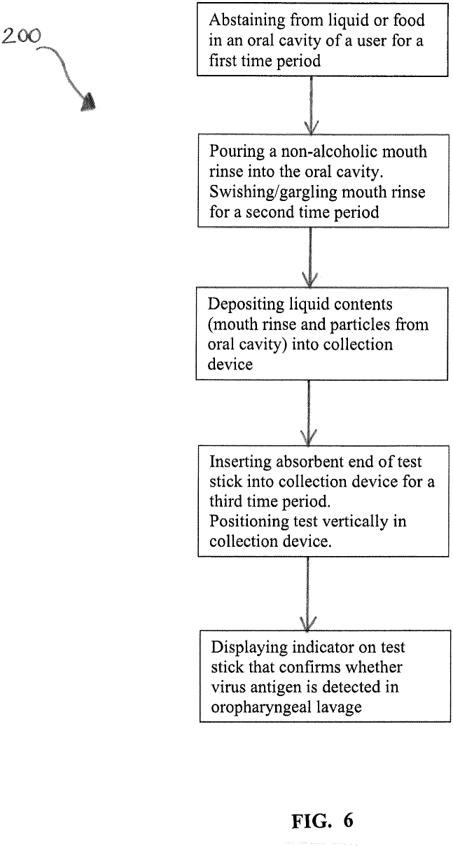

In the embodiment illustrated in FIGS. 6-9, the method 200 of detecting SARS-CoV-2 virus antigen present in an oropharyngeal lavage includes first abstaining from all liquids and foods for a recommended period of time, such as twenty minutes, before performing the test. As with the above method, the method illustrated in FIG. 6 includes preferably abstaining from food or liquid being placed in the oral cavity of the user for that period of time before collecting a sample used for detection. After that first time period, a non-alcoholic mouth rinse is poured into the mouth/oral cavity of the user and moved around the oral cavity by swishing and gargling for a second period of time, for example, one minute. This allows enough time for the mouth rinse to be thoroughly distributed around the inside of the oral cavity and throughout an oropharyngeal space of the user. The mouth rinse should not be spit out of the user’s mouth or swallowed, but rather gargled when the user tilts back and gently blows air through the liquid in the back of the oropharyngeal space. As with the above method, a test stick having first and second ends is used to detect the presence of a virus, for example, or immunization of a virus, from the contents of the user’s mouth.

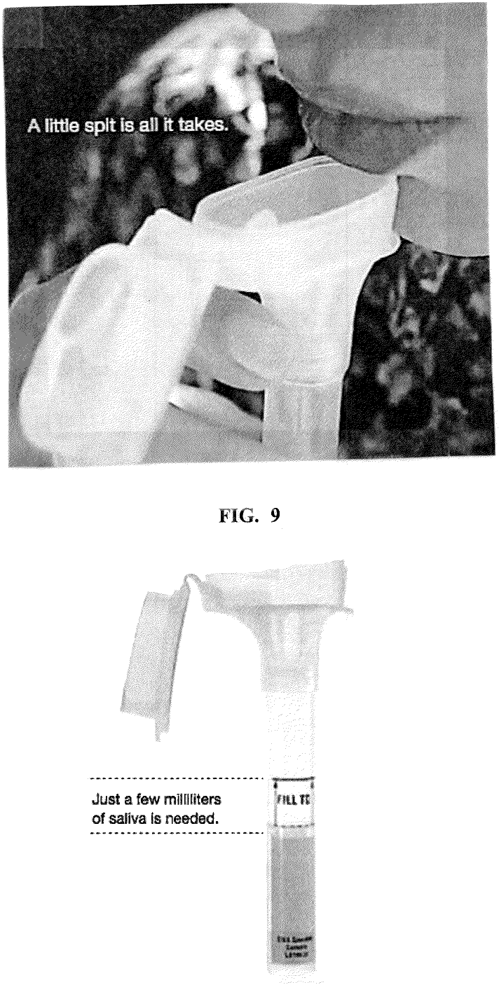

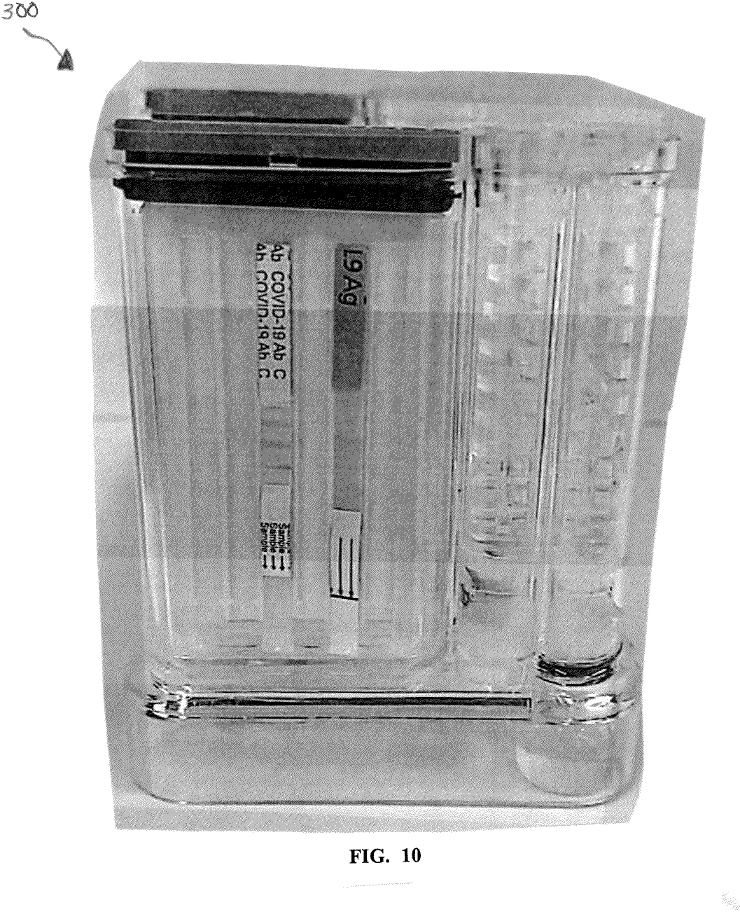

With the embodiment illustrated in FIGS. 6-9, the test stick from this kit 250 may be narrower than that of the test stick 150 of the first method 100. The test stick from this kit 250 is positioned such that the absorbent end is facing downwardly. As shown with method 200, the test stick from this kit 250 is positioned for a fourth period of time in order to register the results of the test and is positioned vertically in the collection device, shown in FIG. 9, as will be further explained below. Once the test results are completed, an indicator reflecting the results of what is being tested for (e.g, SARS-CoV-2 presence, SARS-CoV-2 immunization), the results are displayed on the test stick. In the example illustrated in FIGS. 6-9, the test stick confirms whether SARS-CoV-2 virus antigen is detected in the oropharyngeal lavage.

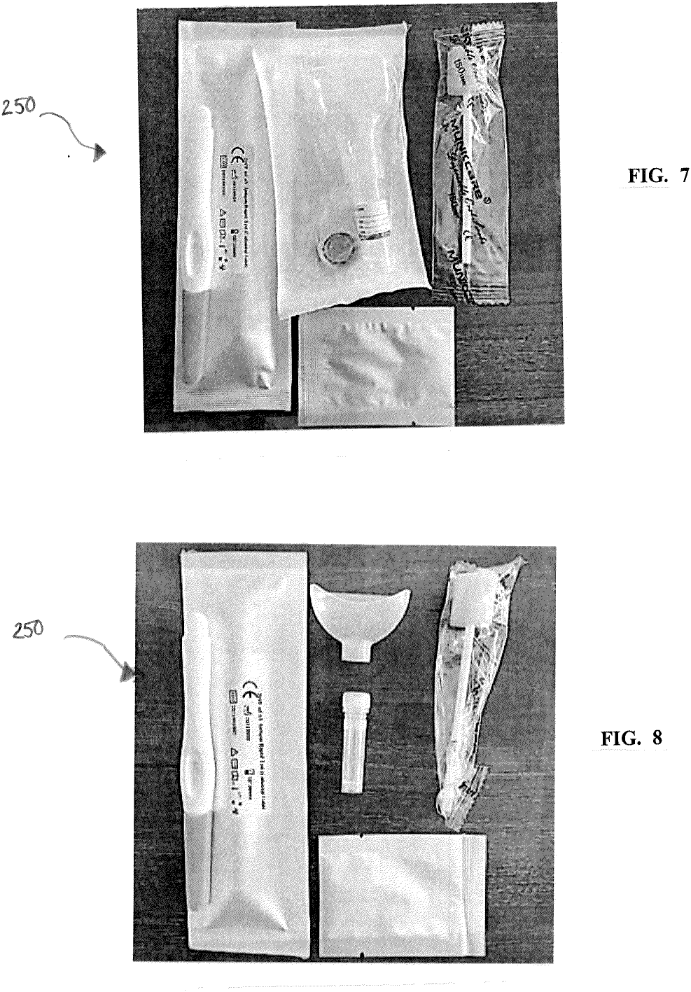

Next, the volume of liquid contents from the oral cavity as obtained from the mouth rinse that also includes particles retrieved from the swishing and gargling of the mouth rinse are deposited into a collection device. With the method illustrated in FIG. 6, the test stick shown in FIGS. 7 and 8 is inserted into the collection device shown in FIG. 9 for detecting capabilities. With this example, the test stick used for testing may be narrower than that used in the above embodiment. The width of the test stick should be configured for being supported in a collection device, such as that shown in FIG. 9. Specifically, an absorbent end of the test stick is inserted into the collection device for a third period of time, such as 10 minutes, and positioned in a vertical position such that the test stick is retained in the collection device wherein the absorbent end of the test stick is facing downwardly. The absorbent end is inserted into the collection device such that the absorbent end is completely immersed in the volume of liquid contents. This allows the absorbent end of the test stick to soak up the contents of the liquid for detection. Finally, an indicator on the test stick displays the results of whether SARS-CoV-2 virus antigen is detected in the oropharyngeal lavage.

Alternatively, the test stick may also display the presence of a variety of markers, e.g., SARS-CoV-2 virus, Herpes simplex virus, Epstein barr virus, Cytomegalovirus, Moraxella catarrhalis, Norocardia sp, Streptococcus mutans, Streptococcus oralis, Eikenella sp. Neisseria sp, and Streptococcus salivarius.

As seen in FIGS. 7 and 8, the test stick has first and second ends and includes an absorbent tab or surface disposed at the first end. The first end includes a removable cap covering the absorbent tab in order to preserve the sterility of the first end until the cap is removed from the first end for the test. The tab is attached to a cartridge disposed within the test stick, wherein the cartridge is configured to detect a virus, such as SARS-CoV-2 virus antigen. The tab is configured for being immersed in liquid, wherein in this embodiment, the tab absorbs at least the mouth rinse and contents of the oral cavity of the user. Once the user swishes the mouth rinse around their oral cavity for the desired period of time, the contents of the user’s mouth, including the mouth rinse and the particles released in the saliva from the mouth rinse are spit into the collection device, as shown in FIG. 9.

Specifically, after the user spits out the contents of their mouth, including the mouth rinse and particles released in the saliva due to the gargling and swishing of the mouth rinse, into the collection device, the absorbent tab is inserted into the collection device. Thus, the collection device should be sized and of a material to retain the volume of liquid dispensed from the user and the absorbent tab. The collection device seen in FIG. 9 is closed at a first end and has an opening at the opposite end, the opening being configured to receive at least the volume of the mouth rinse and contents from the oral cavity of the user. The collection device is further configured to receive the absorbent tab such that the absorbent tab is partially or completely submerged in the volume of liquid spit into the collection device and retained in the collection device when the first end of the test stick is inserted into the collection device.

As with the method illustrated in FIG. 1, with the method illustrated in FIG. 6, after the test stick is positioned vertically for a fourth period of time, such as ten minutes and retained in a vertical position, with the absorbent end facing downwardly, the test results appear on the test stick, confirming whether the antigen being tested for is present or missing from the contents in the collection device.

In the embodiment illustrated in FIG. 10, a device 300 provides a rapid screening test for analytes, such as SARS-CoV-2 virus, Herpes simplex virus, Epstein barr virus, Cytomegalovirus, Moraxella catarrhalis, Norocardia sp, Streptococcus mutans, Streptococcus oralis, Eikenella sp. Neisseria sp, and Streptococcus salivarius. The device includes a vertical flow test strip and well as a chromatographic immunoassay for the simultaneous, qualitative or quantitative detection of analytes in a fluid sample. For example, the fluid sample may include saliva, urine, blood, mucus, water, or fluid extract of a solid or a semi-solid, for example stool or mucus or liquid biopsy. The fluid sample may result from a user pouring a non-alcoholic mouth rinse into their oral cavity, swishing and gargling the non-alcoholic mouth rinse for a period of time to distribute the non-alcoholic mouth rinse around the inside of the oral cavity and throughout an oropharyngeal space of the user. An absorbent stick is then inserted into the mouth of the user or into a collection device to soak up the combination of mouth rinse and contents of the oral cavity that were retrieved from the mouth rinse breaking up by interacting with saliva to activate contents throughout the inside of the mouth, including the inside of the cheeks, the tongue and back of the throat, and to absorb the mouth rinse.

With this embodiment, illustrated in FIG. 10, the device 300 includes at least one membrane test strip in fluid communication with a sample receiving member configured to indicate the presence or absence of at least one analyte above or below a threshold concentration in the fluid sample using a vertical flow chromatographic assay. The device includes a receiving member having an opening to receive a fluid sample. The receiving member may be in fluid communication with other components of the apparatus, for example at least one membrane test strip, sample retention member, and/or an Immunoassay-based fingerprint acquisition pad, through channels, for example tubes, piping, channels molded or carved into the apparatus, or any other suitable structure, made of any suitable material, for example plastic, ceramic, metal, glass, wood, rubber, polymer, fiber-reinforced polymer, or any combination thereof.

The device is configured to include a collection compartment in order to retain at least a volume of liquid dispensed from the user and an absorbent tab to soak up the contents from the volume of liquid. The collection device is closed at a first end and has an opening at the opposite end, the opening being configured to receive at least the volume of the mouth rinse and contents from the oral cavity of the user, and configured to receive the absorbent tab such that the absorbent tab is partially or completely submerged in the volume and retained in the collection device when the first end of the test stick is inserted into the collection device. The test stick is retained in place in the collection device for a period of about 10 minutes in order to register the results of the test. Once the test results are completed, an indicator on the test stick reflecting the results of what is being tested for (e.g, SARS-CoV-2 presence, SARS-CoV-2 immunization), the results are displayed on the test stick or across multiple test sticks in the device.

The term “about” is intended to include the degree of error associated with measurement of the particular quantity based upon the equipment available at the time of filing the application.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The present disclosure includes the following embodiments. The embodiments are refer to preceding embodiments, thus explicitly making logical connections between the embodiments. When a particular feature, structure, or characteristic is described in connection with an embodiment, it is within the ability of one skilled in the art to include such feature, structure, or characteristic in connection with other embodiments whether or not such combination is explicitly described elsewhere in the disclosure.

A method of detecting SARS-CoV-2 virus antigen present in an oropharyngeal lavage, the method including the following steps: abstaining from placing liquid or food in an oral cavity of a user for a brief period of time before collecting a sample, pouring a non-alcoholic mouth rinse into the oral cavity, and swishing and gargling the non-alcoholic mouth rinse for a first period of time to distribute the non-alcoholic mouth rinse around the inside of the oral cavity and throughout an oropharyngeal space of the user, inserting an absorbent end of a test stick into the oral cavity for a second period of time to collect liquid contents from the oral cavity, the liquid contents including the non-alcoholic mouth rinse and particles retrieved from the non-alcoholic mouth rinse, removing the absorbent end of the test stick from the oral cavity and covering the absorbent end with a cap, positioning the test stick in a Flip-Flop vertical position for a third period of time wherein the absorbent end of the test stick is facing upwardly, and displaying an indicator on the test stick that confirms whether SARS-CoV-2 virus antigen is detected in the oropharyngeal lavage.

The method of detecting SARS-CoV-2 virus antigen present in the oropharyngeal lavage of the preceding embodiment, wherein the pretest period of abstaining from liquid or food is at least 20 minutes, the first period of swishing and gargling the non-alcoholic mouth rinse is at least one minute, and the second period of collecting liquid contents is at least one minute.

The method of detecting SARS-CoV-2 virus antigen present in the oropharyngeal lavage of any of the preceding embodiments, wherein the absorbent end of the test stick is inserted under a tongue of the user and held in place for at least one minute without moving the absorbent end, the absorbent end of the test stick being configured to soak up the entire liquid contents of the oral cavity.

The method of detecting SARS-CoV-2 virus antigen present in the oropharyngeal lavage of any of the preceding embodiments, wherein the end of the test stick opposite the absorbent end is inserted into a stand configured to retain the test stick in the vertical position and the third period is at least five minutes.

A method of detecting SARS-CoV-2 virus antigen present in an oropharyngeal lavage, the method including the following steps: abstaining from placing liquid or food in an oral cavity of a user for a period of time before collecting a sample, pouring a non-alcoholic mouth rinse into the oral cavity, and swishing and gargling the non-alcoholic mouth rinse for a first period of time such that the non-alcoholic mouth rinse moves throughout both the oral cavity and an oropharyngeal space of the user, depositing a volume of liquid contents from the oral cavity into a collection device, wherein the volume of liquid contents includes the non-alcoholic mouth rinse and particles retrieved from the non-alcoholic mouth rinse, inserting an absorbent end of a test stick into the collection device for a second period and positioning the test stick in a vertical position such that the test stick is retained in the collection device wherein the absorbent end of the test stick is facing downwardly, and displaying an indicator on the test stick that confirms whether SARS-CoV-2 virus antigen is detected in the oropharyngeal lavage.

The method of detecting SARS-CoV-2 virus antigen present in the oropharyngeal lavage of the preceding embodiment, wherein the period of abstaining from liquid or food is at least 20 minutes, and the first period of swishing and gargling the non-alcoholic mouth rinse is at least 30 seconds.

The method of detecting SARS-CoV-2 virus antigen present in the oropharyngeal lavage of any of the preceding two embodiments, wherein the second period of time is at least ten minutes and the absorbent end of the test stick is inserted into the collection device such that the absorbent end is completely immersed in the volume of liquid contents.

A kit for detecting SARS-CoV-2 virus antigen present in oropharyngeal lavage, the kit comprising: a test stick having a first end and a second end; a non-alcoholic mouth rinse; an absorbent tab disposed at the first end and configured to be inserted into an oral cavity of a user and configured to absorb at least the non-alcoholic mouth rinse and contents of the oral cavity of the user; a removable cap configured to cover said absorbent tab and configured to keep the absorbent tab sterile and dry until said removable cap is removed; a cartridge connected to said absorbent tab and configured to detect the SARS-CoV-2 virus antigen after the non-alcoholic mouth rinse is combined with contents from the oral cavity of the user and absorbed by the absorbent tab; and a stand having an opening configured to receive the second end of the test stick after the combined non-alcoholic mouth rinse and contents from the oral cavity of the user have been discharged onto the absorbent tab.

A kit for detecting SARS-CoV-2 virus antigen present in oropharyngeal lavage, the kit comprising: a test stick having a first end and a second end wherein the first end is configured to be covered by a removable cap to preserve the sterility of the first end until the removable cap is removed from the first end; a non-alcoholic mouth rinse; an absorbent tab disposed at the first end and attached to a cartridge disposed within the test stick, said cartridge configured to detect the SARS-CoV-2 virus antigen after a volume including at least the non-alcoholic mouth rinse combined with contents from the oral cavity of the user; and a collection device having an opening configured to receive at least the volume of the non-alcoholic mouth rinse and contents from the oral cavity of the user, and configured to receive the absorbent tab such that the absorbent tab is submerged in the volume and retained in the collection device when the first end of the test stick is inserted into the collection device.

A kit for detecting SARS-CoV-2 virus antigen present in oropharyngeal lavage, the kit comprising: a sample receiving member having an opening for receiving a fluid sample; a test cartridge member in fluid communication with the sample receiving member, to indicate the presence or absence of at least one analyte in the fluid sample, the test cartridge member comprises a test cartridge chamber and a test cartridge fluid reservoir in fluid communication with the sample receiving member, wherein the test cartridge is inserted into the test cartridge chamber and wherein the at least one test strip is in fluid communication with the test cartridge fluid reservoir; wherein the test cartridge member further includes a cap to secure the test cartridge and at least one of a front set of test strip slots and a back set of test strip slots; and at least one test strip located in the test strip slots, wherein the test cartridge is inserted into the test cartridge member and a plurality of test strips are inserted into the front and back sets of test strip slots and are in fluid communication with the test cartridge fluid reservoir; and an absorbent tab configured for soaking up a volume of mouth rinse and contents from the oral cavity of the user activated by the volume of mouth rinse.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular embodiment disclosed as the best mode contemplated for carrying out this present disclosure, but that the present disclosure will include all embodiments falling within the scope of the claims.

What is claimed is:

1. A method of detecting SARS-CoV-2 virus antigen present in an oropharyngeal lavage, the method including the following steps:
abstaining from placing liquid or food in an oral cavity of a user for a brief period of time before collecting a sample,
pouring a non-alcoholic mouth rinse into the oral cavity, and swishing and gargling the non-alcoholic mouth rinse for a first period of time to distribute the non-alcoholic mouth rinse around the inside of the oral cavity and throughout an oropharyngeal space of the user,
inserting an absorbent end of a test stick into the oral cavity for a second period of time to collect liquid contents from the oral cavity, the liquid contents including the non-alcoholic mouth rinse and particles retrieved from the non-alcoholic mouth rinse,
removing the absorbent end of the test stick from the oral cavity and covering the absorbent end with a cap,
positioning the test stick in a Flip-Flop vertical position for a third period of time wherein the absorbent end of the test stick is facing upwardly, and
displaying an indicator on the test stick that confirms whether SARS-CoV-2 virus antigen is detected in the oropharyngeal lavage.

2. The method of detecting SARS-CoV-2 virus antigen present in the oropharyngeal lavage of claim 1, wherein the pretest period of abstaining from liquid or food is at least 20 minutes, the first period of swishing and gargling the non-alcoholic mouth rinse is at least one minute, and the second period of collecting liquid contents is at least one minute.

3. The method of detecting SARS-CoV-2 virus antigen present in the oropharyngeal lavage of claim 1, wherein the absorbent end of the test stick is inserted under a tongue of the user and held in place for at least one minute without moving the absorbent end, the absorbent end of the test stick being configured to soak up the entire liquid contents of the oral cavity.

4. The method of detecting SARS-CoV-2 virus antigen present in the oropharyngeal lavage of claim 1, wherein the end of the test stick opposite the absorbent end is inserted into a stand configured to retain the test stick in the vertical position and the third period is at least five minutes.

5. A method of detecting SARS-CoV-2 virus antigen present in an oropharyngeal lavage, the method including the following steps:
abstaining from placing liquid or food in an oral cavity of a user for a period of time before collecting a sample,
pouring a non-alcoholic mouth rinse into the oral cavity, and swishing and gargling the non-alcoholic mouth rinse for a first period of time such that the non-alcoholic mouth rinse moves throughout both the oral cavity and an oropharyngeal space of the user,
depositing a volume of liquid contents from the oral cavity into a collection device, wherein the volume of liquid contents includes the non-alcoholic mouth rinse and particles retrieved from the non-alcoholic mouth rinse,
inserting an absorbent end of a test stick into the collection device for a second period and positioning the test stick in a vertical position such that the test stick is retained in the collection device wherein the absorbent end of the test stick is facing downwardly, and
displaying an indicator on the test stick that confirms whether SARS-CoV-2 virus antigen is detected in the oropharyngeal lavage.

6. The method of detecting SARS-CoV-2 virus antigen present in the oropharyngeal lavage of claim 5, wherein the period of abstaining from liquid or food is at least 20 minutes, and the first period of swishing and gargling the non-alcoholic mouth rinse is at least 30 seconds.

7. The method of detecting SARS-CoV-2 virus antigen present in the oropharyngeal lavage of claim 5, wherein the second period of time is at least ten minutes and the absorbent end of the test stick is inserted into the collection device such that the absorbent end is completely immersed in the volume of liquid contents.

8. A kit for detecting SARS-CoV-2 virus antigen present in oropharyngeal lavage, the kit comprising:
a test stick having a first end and a second end;
a non-alcoholic mouth rinse;
an absorbent tab disposed at the first end and configured to be inserted into an oral cavity of a user and configured to absorb at least the non-alcoholic mouth rinse and contents of the oral cavity of the user;
a removable cap configured to cover said absorbent tab and configured to keep the absorbent tab sterile and dry until said removable cap is removed;
a cartridge connected to said absorbent tab and configured to detect the SARS-CoV-2 virus antigen after the non-alcoholic mouth rinse is combined with contents from the oral cavity of the user and absorbed by the absorbent tab; and
a stand having an opening configured to receive the second end of the test stick after the combined non-alcoholic mouth rinse and contents from the oral cavity of the user have been discharged onto the absorbent tab.

9. A kit for detecting SARS-CoV-2 virus antigen present in oropharyngeal lavage, the kit comprising:
a test stick having a first end and a second end wherein the first end is configured to be covered by a removable cap to preserve the sterility of the first end until the removable cap is removed from the first end;
a non-alcoholic mouth rinse;
an absorbent tab disposed at the first end and attached to a cartridge disposed within the test stick, said cartridge configured to detect the SARS-CoV-2 virus antigen after a volume including at least the non-alcoholic mouth rinse combined with contents from the oral cavity of the user; and
a collection device having an opening configured to receive at least the volume of the non-alcoholic mouth rinse and contents from the oral cavity of the user, and configured to receive the absorbent tab such that the absorbent tab is submerged in the volume and retained in the collection device when the first end of the test stick is inserted into the collection device.

10. A kit for detecting SARS-CoV-2 virus antigen present in oropharyngeal lavage, the kit comprising:
a sample receiving member having an opening for receiving a fluid sample;
a test cartridge member in fluid communication with the sample receiving member, to indicate the presence or absence of at least one analyte in the fluid sample, the test cartridge member comprises a test cartridge chamber and a test cartridge fluid reservoir in fluid communication with the sample receiving member, wherein the test cartridge is inserted into the test cartridge chamber and wherein the at least one test strip is in fluid communication with the test cartridge fluid reservoir;
wherein the test cartridge member further includes a cap to secure the test cartridge and at least one of a front set of test strip slots and a back set of test strip slots; and
at least one test strip located in the test strip slots, wherein the test cartridge is inserted into the test cartridge member and a plurality of test strips are inserted into the front and back sets of test strip slots and are in fluid communication with the test cartridge fluid reservoir; and
an absorbent tab configured for soaking up a volume of mouth rinse and contents from the oral cavity of the user activated by the volume of mouth rinse.
